(19) United States
(12) Patent Application Publication
(10) Pub. No.: US 2025/0213377 A1
(43) Pub. Date: Jul. 3, 2025

(54) Apparatus for Stabilizing Body Joints and/or for Supporting Sports Equipment

(71) Applicant: BETTERGUARDS TECHNOLOGY GmbH, BERLIN (DE)

(72) Inventors: Vinzenz BICHLER, Berlin (DE); Oscar BUSCHINGER, Berlin (DE); Elron BORCHARDT, Berlin (DE)

(73) Assignee: BETTERGUARDS TECHNOLOGY GmbH, BERLIN (DE)

(21) Appl. No.: 18/849,598

(22) PCT Filed: Mar. 21, 2023
(86) PCT No.: PCT/EP2023/057189

§ 371 Date: Sep. 23, 2024

(30) Foreign Application Priority Data
Mar. 21, 2022 (DE) 10 2022 106 595.2

(51) Int. Cl. A61F 5/01

(52) U.S. Cl. CPC A61F 5/0102; A61F 2005/0169; A61F 2005/0197

(57) ABSTRACT

An apparatus for stabilizing body joints and/or for supporting sports equipment, comprising: a receptacle, wherein the receptacle is filled with a filling medium, a first body for interacting with the filling medium, wherein the first body is arranged in a displaceable manner in the receptacle, a force transmission body for transmitting an external force to the first body, a second body for interacting with the filling medium, which second body is arranged in a displaceable manner in the receptacle, wherein the second body can be elastically coupled to the first body via a coupling element, wherein at least the first body has at least one passage opening through which the filling medium can flow, wherein the second body is displaceably received within the passage opening of the first body, wherein the first body comprises a valve seat in the region of the passage opening and the second body forms a valve body, so that a flow of the filling medium through the passage opening can be permitted or prevented depending on the valve position.

### TECHNICAL FIELD

The present disclosure relates to an apparatus for stabilizing body joints and/or for supporting sports equipment, which apparatus comprises a receptacle which is filled with a filling medium, a first body for interacting with the filling medium, which first body is arranged in a displaceable manner in the receptacle, a force transmission body for transmitting an external force to the first body, and a second body for interacting with the filling medium, which second body is arranged in a displaceable manner in the receptacle.

### DESCRIPTION OF THE RELATED ART

It is known to stabilize body joints, muscles and tendons by means of apparatuses which permit adaptive movement limitation. Furthermore, it is known to provide sports equipment which can be subjected to movements-related loads with adaptive movement limitation apparatuses.

Inter alia, the adaptive behaviour of such apparatuses is achieved in that two bodies move relative to one another, wherein a filling medium is located between the bodies. In this case, one body of the apparatus can form a receptacle which is filled with the filling medium. The other body can form a pull-out body which is movably arranged in the receptacle. In the region between the receptacle and the pull-out body, the filling medium can flow when the two bodies move relative to one another. The flow speed of the filling medium depends decisively on the cross-sectional area perpendicular to a relative displacement direction of the receptacle and of the pull-out body. This cross-sectional area which is available for the flow for the filling medium is also referred to as hydraulic diameter and ultimately is decisive for the reactive behaviour of the apparatus in the event of an external action of force. Thus, the resistance which the apparatus opposes to external forces can be fixed by the selection of the hydraulic diameter. The apparatuses can be fixed between two body sites of a user or two elements of a sports equipment which are movable relative to one another.

If physiological forces, that is to say forces which are not critical for the corresponding body part or component to be stabilized, are introduced into the apparatus via the two body sites of the user, a corresponding relative movement of the receptacle and of the pull-out body and therefore a movement of the body part to be stabilized is permitted in accordance with the hydraulic diameter in the apparatus.

If, by contrast, forces which are not physiological, that is to say forces which are critical for the corresponding body part or component to be stabilized, are introduced into the apparatus, a relative movement between the pull-out body and the receptacle is only possible with a very high expenditure of force on account of the hydraulic diameter.

Apparatuses for stabilizing body joints which permit an adaptive behaviour as a function of the intensity of an acting force are known from EP 3 238 670 A1 and WO 2020/115227 A1.

The apparatuses each comprise a receptacle which is filled with a filling medium, a first body for inter-acting with the filling medium, wherein the first body is arranged in a displaceable manner in the receptacle, and a force transmission body for transmitting an external force to the first body. A second body for interacting with the filling medium is arranged in a displaceable manner in the receptacle, wherein the second body is elastically coupled to the first body via a coupling element. Furthermore, the second body has a passage opening through which the filling medium can flow. The passage opening in the second body provides a hydraulic diameter for the filling medium, through which hydraulic diameter the filling medium can flow as long as there is a spacing between the first body and the second body.

In this case, the first body forms a valve body and the second body forms a valve seat, so that a flow of the medium through the passage opening can be permitted or prevented depending on the valve position.

External forces acting on the first body can be transmitted to the second body via the coupling element. Accordingly, the first body is capable of pushing and/or pulling the second body through the filling medium by means of the coupling element.

In this case, the coupling element is configured such that when an external force acts on the first body, in the region of a physiological speed, it transmits a force to the second body, so that the latter can be moved together with the first body through the filling medium.

If the force acting on the second body via the first body and the coupling element leads to critical relative displacement speeds in the apparatus, that is to say to non-physiological speeds, the coupling element yields, as a result of which the first body moves towards the second body. As a result, the hydraulic diameter is reduced until the valve formed by the two bodies is closed.

If a hydraulic diameter through which the filling medium can flow is no longer available, the first body and the second body cannot be moved further through the filling medium. The apparatus blocks.

It has been shown that the switching behaviour of the apparatuses explained above between an open and a closed valve position is also influenced substantially by the frictional forces which occur between the second body and the inner surface of the receptacle. The frictional force between the second body and the inner surface of the receptacle counteracts the forces acting indirectly on the second body via the first body and the coupling element, as a result of which the closing behaviour of the valve is influenced directly.

### OVERVIEW

The present disclosure provides an apparatus for stabilizing body joints or for supporting sports equipment.

Correspondingly, an apparatus for stabilizing body joints and/or for supporting sports equipment is described, comprising: a receptacle, wherein the receptacle is filled with a filling medium, a first body for interacting with the filling medium, wherein the first body is arranged in a displaceable manner in the receptacle, a force transmission body for transmitting an external force to the first body, a second body for interacting with the filling medium, which second body is arranged in a displaceable manner in the receptacle, wherein the second body can be coupled elastically to the first body via a coupling element, wherein at least the first body has at least one passage opening through which the filling medium can flow. According to the disclosure, the second body is displaceable received within the passage opening of the first body, wherein the first body comprises a valve seat in the region of the passage opening and the second body forms a valve body, so that a flow of the filling medium through the passage opening can be permitted or prevented de-pending on the valve position.

By means of the apparatus described above, it is possible to decouple the direct switching behaviour between the first body and the second body from the influence of the frictional force between the first body and the receptacle. If the first body is moved through the receptacle by means of the force transmission body, the filling medium flows through the passage opening and thereby flows around the second body. Depending on the forces which act on the second body as a result of the filling medium flowing around, the second body can be moved towards the valve seat against a clamping force of the coupling element until the valve is completely closed.

Thus, the relative movement between the first body and the second body depends primarily on the clamping force of the coupling element and the speed of the filling medium flowing around or the flow force of the filling medium on the second body. Frictional forces which act between the first body and an inner surface of the receptacle have no direct influence on the relative movement between the first body and the second body. In particular, possible fluctuations of the frictional forces which occur between the first body and the inner surface of the receptacle do not have an effect on the switching or closing behaviour of the second body with respect to the valve seat of the first body. Fluctuations can occur, for example, in the form of friction-causing material pairings, static friction or friction caused by a curvature of the receptacle or a bending of the apparatus.

The term “passage opening” means a passage, at least through the first body, which provides a passage path or flow path for the filling medium. The passage opening can be formed in a tunnel-shaped, channel-shaped or in the form of a passage of another type.

The term “force transmission body” in the present case generally comprises an elongate, flexible, optionally elastic element which can have the form of an individual fibre, a fibre strand, a wire, a cord, a cable, a textile fabric with a limited width and fixed bilateral selvedge or the like. Alternatively, the force transmission body can also be formed as a rod element which is resistant to tension and has shear properties. In this case, the rod element is can be formed in one piece with the first body.

In an embodiment, the passage opening has at least one guide section extending in a relative displacement direction of the second body with respect to the first body. As a result, the second body can be guided in the relative displacement direction. The guide section defines an inner surface of the passage opening of the first body, along which inner surface the second body can be moved with respect to the first body between a starting position and a closed position of the apparatus.

In a further embodiment, the inner surface of the passage opening has a guide. As a result, the second body can be held centrally with respect to the cross section of the passage opening. In this way, it can be ensured that a predefined spacing is provided between the inner surface of the passage opening and the second body, through which predefined spacing the filling medium can flow. This predefined spacing forms the hydraulic diameter of the apparatus.

In a development, the valve seat limits a relative displacement path of the second body with respect to the first body in the relative displacement direction.

In a development, the relative dimensions of the second body with respect to the pas-sage opening are designed such that in the open valve position a fluid flow of the filling medium can be permitted in the region between the second body and the inner surface of the passage opening. As a result, the second body can be flowed around by means of the filling medium when an external force acts on the apparatus. The flow around the second body can lead, as a function of the magnitude or the speed at which an external force acts on the apparatus, to a flow speed or a flow force of the filling medium which moves the second body towards the valve seat of the first body.

In a further embodiment, the second body is held in an open valve position by the coupling element in a starting position of the apparatus. As a result, it can be ensured that a flow path through the passage opening of the first body is provided for the filling medium in a starting position of the apparatus.

In a configuration, the coupling element is configured such that when physiological forces or speeds act on the apparatus the second body can be held in the open valve position by the coupling element against a flow direction of the filling medium, and when non-physiological forces or speeds act on the apparatus the flow force of the filling medium can displace the second body into the closed valve position against a clamping force of the coupling element and hold it therein.

In a development, the second body is received in the passage opening of the first body such that filling medium can flow around at least one region of the surface of the second body in an open valve position. As a result, it is possible that flow forces acting on the second body can occur as a result of the filling medium flowing around the second body. If the flow forces acting on the second body are greater than a clamping force of the coupling element, the second body can be moved in the direction of the valve seat of the first body until the passage opening of the first body is completely closed.

In a further embodiment, the surface of the second body around which flow can pass extends from a first surface section, which faces at least one first end opening of the passage opening, to a second surface section, which faces at least one second opening or end opening of the passage opening. A flow around the second body from the first surface section thereof to the second surface section thereof can lead, as a function of the flow speed or inflow speed of the filling medium, the nature of the filling medium and the shape of the second body, to a greater pressure force acting on the first surface section, that is to say the inflow section, than on the second surface section. The force resulting from the pressure difference acts here in the direction of the flow of the filling medium and, in the event of a flow in the direction of the valve seat, is responsible for the displacement of the second body towards the valve seat.

In a configuration, the apparatus comprises a first body with a multiplicity of valve seats and a number of second bodies corresponding to the number of valve seats. The valve seats can have passage openings of different sizes and/or be arranged in series. The valve seats arranged in series are arranged in a stepped manner depending on the size of their passage opening, so that the passage openings of the valve seats decrease from the first end opening of the first body in the direction of the second end opening of the first body. Analogously, the surfaces of the second bodies are each adapted to the dimension of the unblocking passage opening of the corresponding valve seat. As a result, a stepped closing of the valves can be made possible.

In a further embodiment, the second body at least partially has a convex incident flow profile or a conical incident flow profile. As a result, the effect of a pressure difference between the compressive forces acting on an incident flow side and on a side of the second body facing away from the flow can be promoted. Here, the side facing away from the incident flow forms the side of the second body which faces towards the valve seat.

In a development, the second body is spherical.

For the stabilization, the state of the closed valve position is triggered in a controlled and targeted manner. For this purpose, it is advantageous to keep the influence of the friction on the functionality of the second body as low as possible, with the result that a spherical shaping of the second body is advantageous. According to Stokes's law of friction, the friction is proportional to the radius of the sphere, which gives rise to a further advantage of a compact de-sign. Furthermore, the radially symmetrical shaping of a sphere has the advantage that a constant flow profile is provided over the entire surface, with the result that the closing of the valve is independent of a possible rotation of the second body.

In a further embodiment, the first body comprises a sealing section, such as, for example, an adaptive sealing section, which is arranged circumferentially between the first body and the inner surface of the receptacle in order to seal the first body with respect to the receptacle in the closed valve position.

By means of the sealing section, it can be ensured that the filling medium is guided through the passage opening of the first body in an open valve position in order to pass from one side to the other side of the first body within the receptacle. In this case, the sealing section makes contact with the inner surface of the receptacle and permits a relative movement of the first body with respect to the receptacle. This relative movement is counteracted merely by a frictional force which results from the contact of the sealing section and the inner surface of the receptacle.

As a result of the arrangement of the sealing section between the first body and the inner surface of the receptacle, frictional forces which are produced do not act directly on the second body, which acts as a valve body. Thus, the closing behaviour between the second body and the valve seat is influenced substantially by the pressure difference resulting from the flow around the second body.

In particular, possible fluctuations of the frictional forces which occur between the sealing section and the inner surface of the receptacle do not have an effect on the switching or closing behaviour of the second body with respect to the valve seat of the first body. The sealing section may also be formed as an O-ring.

In a development, the sealing section comprises a positioning part, wherein the passage opening is formed at least partially by the positioning part, and wherein the second body can be elastically coupled to the positioning part via a coupling element.

As a result, the positioning part can be configured correspondingly to the first body, such that the positioning part, instead of the first body, elastically couples the second body via the coupling element and has at least one passage opening, so that the filling medium can flow through said passage opening when the valve position is open. Furthermore, the positioning part comprises the valve seat in the region of the passage opening, so that the second body, the guide section of which is provided by the first body, can permit or prevent the flow of the filling medium depending on the valve position. As a result, the first body can be configured more simply and thus more cost-effectively. The connection between the positioning part and the first body can be provided in a form-fitting manner, for example by means of welding, screwing or adhesive bonding, wherein welding merely has to be carried out in the region of the coupling element, with the result that the guide section of the second body is not influenced by the connection.

Furthermore, the positioning part can be shaped such that the second body is displaceable received within the passage opening of the positioning part, as a result of which an advantageous guidance of the second body is achieved and low manufacturing tolerances of the positioning part are possible. The connection of the positioning part to the first body can be carried out in a form-fitting manner, in particular by welding. This configuration can also be used for supporting sports equipment, for example shoes, in particular in the case of lacing, the shoelaces and an adaptive sole which has, for example, a speed-dependent and/or load-dependent bending stiffness.

In an embodiment, the force transmission body or the first body has an end stop, wherein the force transmission body has at least one recess from the end stop in the movement direction up to a cross-sectional widening or the first body has at least one recess from the end stop in the movement direction up to a cross-sectional widening and/or the first body has at least one recess extending from the valve seat along the movement direction in the direction of the force transmission body, so that the filling medium can flow through the at least one recess.

This configuration permits a particularly compact shaping of the first body and of the force transmission body. As a result, the distances between the inner surface of the receptacle and first body and the inner surface of the receptacle and the force transmission body are sufficiently large, so that a bending of the apparatus or curvature of the receptacle along the movement direction is possible. Thus, the apparatus can better follow the course of different shapes. Furthermore, the at least one recess forms at least one passage opening for the filling medium and permits a form-fitting or force-fitting connection of the body having the at least one recess to a body limiting the passage opening.

In a further embodiment, the adaptive section comprises a sealing lip, wherein the sealing lip can be pressed against the inner surface of the receptacle by means of the filling medium in the closed valve position. An increased pressure acting on the apparatus in the closed valve position increases the force with which the sealing lip is pressed against the inner surface of the receptacle.

In a development, the first body divides a cavity of the receptacle into a first chamber and a second chamber, wherein the passage opening can provide a flow of the fluid medium between the first chamber and the second chamber in an open valve position.

### BRIEF DESCRIPTION OF THE FIGURES

Embodiments of the disclosure are explained in more detail by the following description of the figures. In the figures:

FIG. 1A schematically shows a perspective view of an apparatus for stabilizing body joints and/or sports equipment,

FIG. 1B schematically shows a lateral sectional view of the apparatus from FIG. 1A in an initial state,

FIG. 2A schematically shows a detail view of the apparatus according to FIG. 1B in a starting position,

FIG. 2B schematically shows a detail view of the apparatus according to FIG. 1B in a blocking position,

FIG. 3 schematically shows a partial detail of a sectional view of a modified apparatus,

FIG. 4A schematically shows a partial detail of a sectional view of an alternative embodiment of an apparatus,

FIG. 4B schematically shows a partial detail of a sectional view of an alternative embodiment of an apparatus,

FIG. 5 schematically shows a sectional view of an apparatus without sealing lip,

FIG. 6A shows a partial detail of a schematic sectional view of an alternative embodiment of an apparatus with recesses in the first body,

FIG. 6B schematically shows a sectional view along a plane perpendicular to the movement direction of the apparatus shown in FIG. 6a,

FIG. 6C shows a partial detail of a first body formed in one piece and force transmission body with stop,

FIG. 6D shows a sectional view of a positioning part, coupling element and second body,

FIG. 6E shows a partial detail of a schematic sectional view of an alternative embodiment of an apparatus with recesses in the first body, and

FIG. 7 schematically shows a sectional view of a positioning part in an alternative embodiment.

### DETAILED DESCRIPTION OF EXEMPLARY EMBODIMENTS

Exemplary embodiments are described below with reference to the figures. In this case, identical, similar or identically acting elements are provided with identical reference symbols in the different figures. In order to avoid redundancies, a repeated description of these elements is partially dispensed with.

The function of the present apparatus is described below with reference to use in the sports sector, wherein use in the work clothing sector, for example in work boots, is also conceivable. In this case, the apparatus is used for damping the movement of two points of a body which can move relative to one another. Such a body can be, for example, a sports shoe which, coupled to the present apparatus, can counteract a bending movement. However, the present apparatus is not restricted to the fields of use described herein. Thus, it can also be arranged between two body parts of a living being in order to damp a corresponding body movement. Alternatively, the apparatus can also be used in other everyday objects in which abruptly rising forces between a body or an object are intended to be damped. Furthermore, the apparatus in the sports shoe sector can also be used for lacing, in particular the shoelaces and in the form of an adaptive sole.

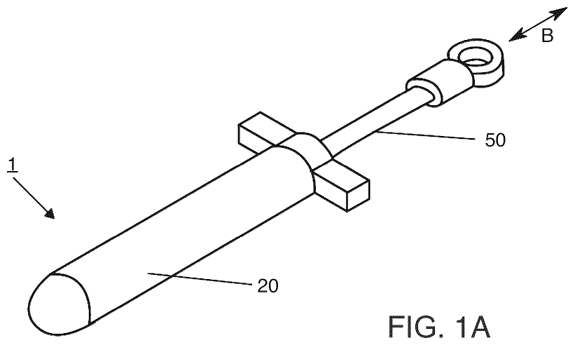

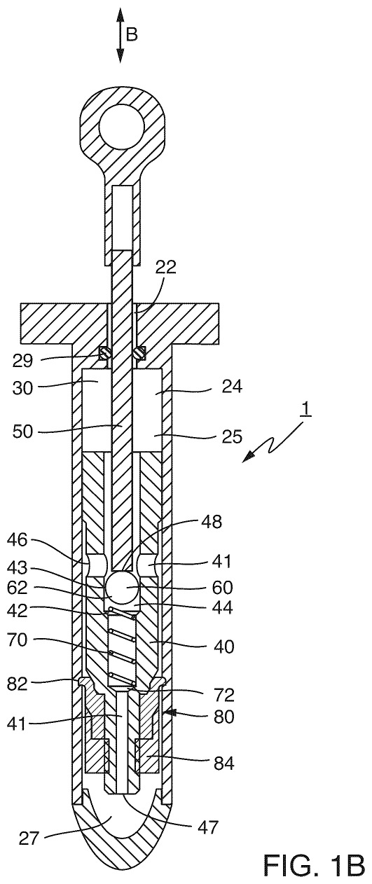

FIG. 1A shows a perspective view of an apparatus 1 for stabilizing body joints and/or sports equipment. A force transmission body 50 protrudes from a cylindrical receptacle 20. In this case, the receptacle 20 can be fastened to a body point of a user or sports equipment such as, for example, a sports shoe. The end of the force transmission body 50 which lies outside the apparatus 20 can be fastened to a second body point. The first body point and the second body point are distinguished in that they are subjected to abrupt relative movements with respect to one another. The direction B represents the movement direction of the apparatus. Alternatively, the receptacle can also be of cuboidal form.

FIG. 1B shows a sectional view of the apparatus 1 which is in an initial state. The receptacle 20 has an opening 22 through which a force transmission body 50 protrudes into the interior 24 of the receptacle 20. If the body point to which the receptacle 20 is fastened moves relative to the body point at which the force transmission body 50 is arranged, the force transmission body 50 moves relative to the receptacle 20. In particular, the force transmission body 50 can move further into the receptacle 20 or further out of the receptacle 20 in a movement direction B.

Arranged in the region of the opening 22 are sealing inserts 29 which seal off the interior 24 of the receptacle 20 from the surroundings, with the result that the filling medium 30 can be held in the interior 24 of the receptacle 20.

The receptacle 20 of the apparatus 1 is produced from stainless steel. Alternatively, the receptacle can also be produced from plastic. Inter alia, fibre-reinforced plastics can also be used. Alternatively, the receptacle can also be produced from other metals such as, for example, aluminium or magnesium. Furthermore, the receptacle can also be produced from ceramic.

The force transmission body 50 is formed by a cable made from wire and extends from a first body 40 through a first chamber 25 of the receptacle 20 and finally through the opening 22 of the receptacle 20. Alternatively, the force transmission body 50 can be formed in the form of a rod element made from plastic. Furthermore, the force transmission body 50 can also be of fibrous form, in particular from plastic fibres such as, for example, glass fibre, carbon fibre and the like or from natural fibres such as, for example, hemp fibre, flax fibre and the like. Furthermore, the force transmission body 50 can also be produced from metal such as, for example, aluminium, magnesium or stainless steel.

An interior 24 of the apparatus 20 is filled with a filling medium 30. The filling medium 30 is a Newtonian fluid such as, for example, silicone oil. Alternatively, dilatant fluids can also be used as filling medium. Furthermore, a shear-thickening plastic can also be used. In this case, the plastic is present in powder form. Furthermore, sand can also be used as filling medium.

Furthermore, arranged in the interior 24 of the apparatus 20 is a first body 40 which is movable in the movement direction B relative to the receptacle 20 by the filling medium 30. In the present embodiment, the first body 40 is pressed with the force transmission body 50, with the result that a force originating from the force transmission body 50 can be transmitted to the first body 40. Alternatively, the first body can also be welded, adhesively bonded, formed in one piece or coupled in another way to the force transmission body 50.

The first body 40 is produced from plastic. Alternatively, the first body can also be produced from a metal such as, for example, aluminium, magnesium or steel.

The cross section of the first body 40 is always smaller than the cross section of the receptacle 20, with the result that the filling medium 30 can flow at least partially on the outer circumferential surface of the first body 40 relative to the receptacle 20.

Furthermore, the first body 40 comprises a passage opening 41 through which the filling medium 30 can flow. If the first body 40 is correspondingly moved relative to the receptacle 20 by a force originating from the force transmission body 50, the filling medium 30 can flow through the passage opening 41 of the first body 40 between the first chamber 25 and the second chamber 27. Accordingly, the passage opening 41 defines a hydraulic diameter for the filling medium 30 through the first body 40.

The fluid connection between the first chamber 25 and the second chamber 27 of the receptacle 20 into a starting position of the apparatus 1 is described below. If the force transmission body 50 is moved out of the receptacle 20, the filling medium 30 can first of all flow on the way from the first chamber 25 to the second chamber 27 through the first hydraulic diameter between the outer circumferential surface of the first body and the inner circumferential surface of the receptacle 20. Subsequently, the filling medium 30 flows through a lateral opening, i.e. a first end opening 46, in the first body 40 into the passage opening 41. Finally, the filling medium 30 can flow from a second end opening 47 of the passage opening 41 into the second chamber 27 of the receptacle 20.

Furthermore, arranged in the passage opening 41 of the first body 40 is a second body 60 which is movable relative to the first body 40 in the movement direction B along a guide section 43. According to FIG. 1B, the second body 60 is of spherical design and has a diameter which is slightly smaller than the diameter of the inner circumference of a guide surface 44 of the passage opening 41. The spacing between the second body 60 and the inner guide surface 44 forms the actual hydraulic diameter of the apparatus 1 through which the active medium 30 can flow and thus can pass the second body 60.

Alternatively, the second body can also have other geometries such as, for example, a partially convex profile or a conical profile and the like. The second body 60 is produced from plastic. Alternatively, the second body can also be produced from a metal such as, for example, aluminium.

The guide section 43 of the passage opening 41 has an end stop 48 for a starting position of the second body 60. According to FIG. 1B, the end stop 48 is formed by the force transmission body 50 which is press-fitted to the first body 40. Opposite the end stop 48, the guide section 43 is limited by a cross-sectional tapering of the passage opening 41. The cross-sectional tapering in the passage opening 41 forms a valve seat 42. The second body 60 acts as a valve body which blocks the passage opening 41 on contact with the valve seat 42.

Furthermore, arranged in the passage opening 41 is a coupling element 70 in the form of a spring which extends between a spring seat 72 within the first body 40 and the second body 60 and couples the first body 40 to the second body 60. The coupling element 70 is subjected to a pre-stress by means of which it can hold the second body 60 in the starting position, i.e. pressed against the end stop 48. According to the present embodiment, the coupling element 70 is held on the second body 60 in the starting position on account of the pre-stress of the coupling element 70. That is to say that the second body 60 bears merely against the coupling element 70.

Alternatively, the coupling element 70 and the second body 60 can also be permanently connected to one another or even formed in one piece. The coupling element 70 is then capable of holding the second body 60 in a starting position, with the result that the above-described end stop 48 is not required in such an embodiment.

According to the present embodiment, the coupling element 70 is additionally configured to hold the second body 60 centrally in the radial direction of the passage opening 41. As a result, it can be ensured that a defined spacing can be provided between the guide surface 44 and the second body 60 in the region of the guide section 43. Alternatively, a guide 45 can also be provided on the guide surface 44, which guide holds the second body 60 centrally with respect to the longitudinal axis of the passage opening 41.

The first body 40 has, on its outer circumferential surface, an adaptive section 80 which is configured to seal against the inner circumferential surface of the receptacle 20. In this case, the adaptive section 80 is positioned on the first body 40 such that it is arranged between the two end openings 46, 47 of the passage opening 41, wherein the first end opening 46 is fluidically assigned to the first chamber 25 and the second end opening 47 is fluidically assigned to the second chamber 27. Thus, the adaptive section 80 prevents a fluid connection from the first chamber 25 to the second chamber 27 on the outer circumferential surface of the first body 40. The only fluid connection between the first chamber 25 in the second chamber 27 is provided by the passage opening 41.

If the force transmission body 50 moves out of the receptacle 20 in the movement direction B, a part of the active medium 30 builds up on the adaptive section 80 and presses it against the inner circumferential surface of the receptacle 20. In the embodiments according to FIG. 1B, this effect is reinforced by a sealing lip 82 on the adaptive section 80.

According to FIG. 1B, the adaptive section 80 is held on the first body 40 by means of a positioning part 84. The positioning part 84 is designed in the form of a screw nut which is screwed onto an external thread on an end section of the first body 40. Alternatively, the adaptive section 80 can also be fastened to the first body 40 by means of a snap ring, for which purpose a corresponding groove for receiving the snap ring can be provided in the end section of the first body 40.

The apparatus 1 shown in FIGS. 1A and 1B is designed for tensile loads. That is to say, for loads which result from a movement apart of the body point to which the receptacle 20 is fastened and of the body point to which the force transmission body 50 is fastened. If the force transmission body 50 is pulled out of the apparatus 1, it pulls the first body 40 with it, as a result of which there is a flow of the active medium 30 from the first chamber 25 along a partial section of the outer circumferential surface of the first body 40, through the passage opening 41 past the second body 60 towards the second chamber 27.

The principle of the apparatus 1 described in FIGS. 1A and 1B can be used in the same way for an apparatus designed for compressive loading. In this case, the force transmission body 50 is formed in the form of a compression rod which is connected to the first body on the side of the second chamber. Accordingly, the exit of the force transmission body 50 from the receptacle is also located on the other side of the receptacle in comparison with FIGS. 1A and 1B.

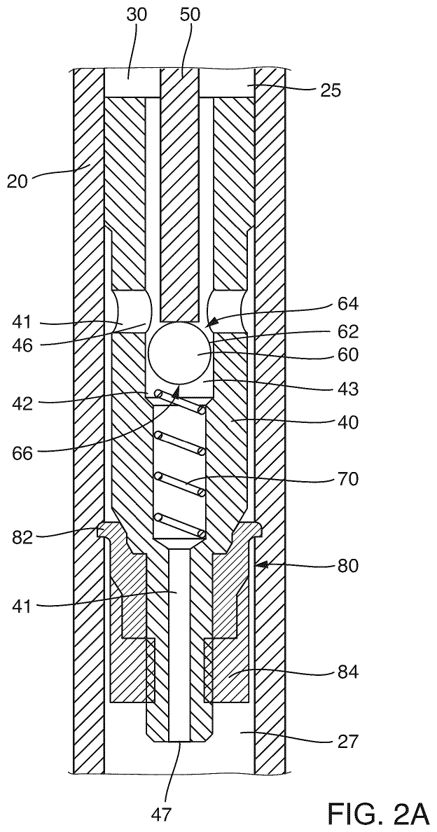

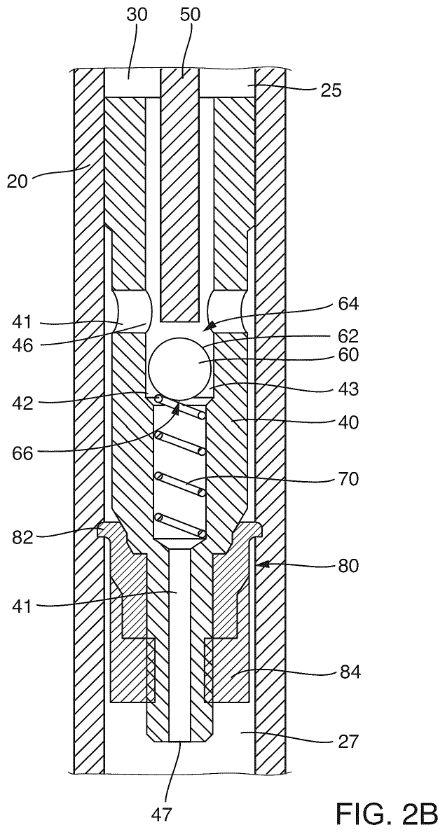

FIGS. 2A and 2B show detail views of the apparatus 1 with reference to which the function of the de-vice 1 is described in more detail. If a force acts in the region of a physiological speed and if the force transmission body 50 is pulled out of the receptacle 20, the active medium 30 flows from the first chamber 25 along the outer circumferential surface of the first body 40, via the end opening 46 into the passage opening 41, past the second body 60 in the interior of the passage opening 41, and finally via the end opening 47 of the passage opening 41 into the second chamber 27.

The surface of the second body 60 can be divided into a first surface section 64, which is oriented toward the first chamber 25, and into a second surface section 66, which is oriented toward the second chamber 27. A flow around the second body 60 from the first surface section 64 thereof to the second surface section 66 thereof can lead, as a function of the flow speed or inflow speed of the filling medium 30, the nature of the filling medium 30 and the shape of the second body, to a greater pressure force acting on the first surface section, that is to say the inflow section, than on the second surface section. The force resulting from the pressure difference acts here in the flow direction of the filling medium 30 and, in the event of a flow in the direction of the valve seat 42, can bring about a displacement of the second body 60 towards the valve seat 42.

The clamping force of the coupling element 70—here the spring strength of a spring-is selected such that the coupling element 70 deflects only when a threshold value of the pressure force acting on the second body 60 is reached. In the event of a resulting pressure force below the threshold value, no relative movement of the second body 60 with respect to the first body 40 takes place. In the event of a pressure force above the threshold value, the spring begins to deflect, as a result of which the second body 60 moves towards the valve seat 42 of the second body 40 until the passage opening 41 is completely closed, as shown in FIG. 2B. In this state, the hydraulic diameter of the apparatus is zero. Thus, there is no longer a hydraulic diameter through which the filling medium 30 can flow between the first chamber 25 and the second chamber 27.

By selecting comparatively low spring strengths, for example 0.05 Nmm to 1 Nmm, in combination with the pre-stress of the spring, a comparatively fast reaction time of the apparatus of approximately 20 ms, i.e. a fast closing of the passage opening 41, can be realized. A flow of the filling medium 30 from the first chamber 25 into the second chamber 27 is no longer possible, with the result that the first body 40 can no longer be moved relative to the receptacle 20. In this state, the apparatus 1 permits no further movement between two body points to be supported/damped.

For example, the threshold value of the pressure force can be 4.5 N and the system can immediately completely close in the event of a jump in a resistance force from 4.5 N to 5 N.

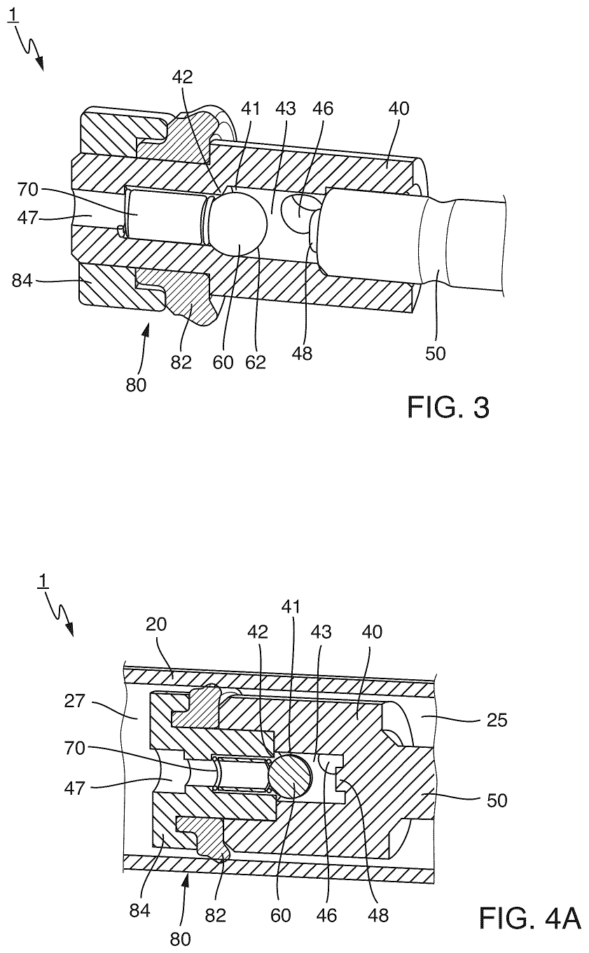

FIG. 3 shows an alternative embodiment which describes a modification of the apparatus 1 described above.

In contrast, in this case, the first body 40 and the force transmission body 50 are produced from plastic by means of an injection-moulding method, as a result of which smaller component dimensions are possible taking into account the manufacturing tolerances.

In the state illustrated, the second body 60 is pressed onto the valve seat 42, that is to say blocks the passage opening 41. In the normal state which is not shown, the coupling element 70 can press the second body 60 against the end stop 48 of the force transmission body 50.

Furthermore, it is possible to arrange additional guide pins in the guide section 43. Said guide pins serve for centring the second body 60 and for compensating manufacturing tolerances, in particular of the spring as coupling element 70.

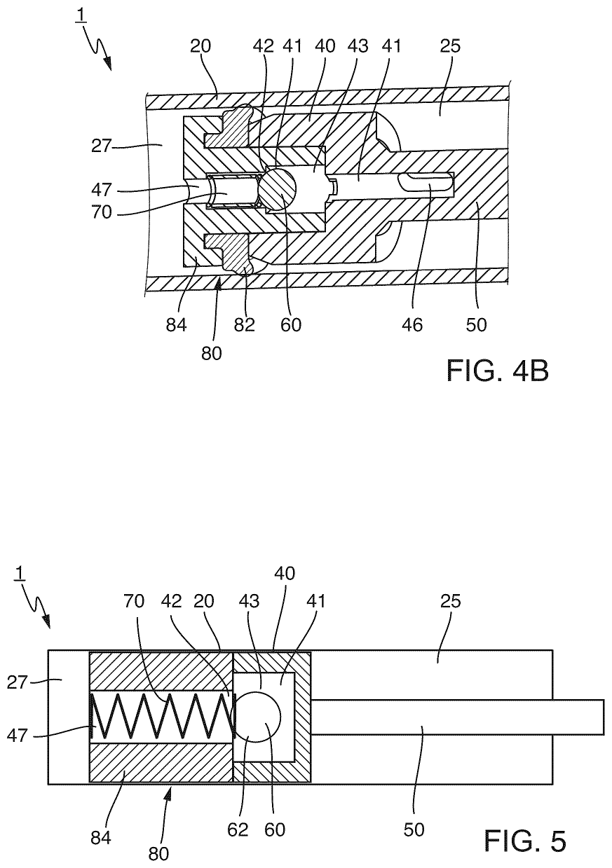

An alternative embodiment of an apparatus 1 is shown below in FIG. 4a.

In this case, a force transmission body 50 and a first body 40 are formed in one piece. As a result of the component reduction, a space-saving and cost-effective embodiment is achieved.

Furthermore, the first body 40 comprises an adaptive sealing section 80, wherein the passage opening 41 is formed at least partially by the positioning part 84, so that the filling medium 30 can flow through the passage opening. The second body 60 can be elastically coupled to the positioning part 84 via a coupling element 70, in this case, in the present exemplary embodiment, the coupling element 70 is a spring. The valve seat is arranged in the region of the passage opening 41, so that the second body 60, the valve body, can permit or prevent the flow of the filling medium 30 through the passage opening 41 depending on the valve position.

The one-piece shaping of the force transmission body 50 and of the first body 40 has a first end opening 46 in the region of the first body 40. The passage opening 41 in the first body 40 has a guide section 43 extending in a relative displacement direction of the second body 60 with respect to the first body 40, in order to guide the second body 60 in the relative displacement direction.

Advantageously, in this configuration, no welding is carried out in the region of the guide section 43.

FIG. 4b shows a perspective view of an embodiment, modified with respect to FIG. 4a, of an apparatus 1 with a force transmission body 50 formed in one piece and first body 40.

In contrast to the embodiment in FIG. 4a, a first end opening 46 is arranged in the region of the force transmission body 50, so that the passage opening 41 is formed partially by the force transmission body 50 and a flow of the filling medium 30 can be permitted or prevented depending on the valve position.

The second body 60 is displaceable received within the region of the passage opening 41 formed by the positioning part 84. For this purpose, the passage opening 41 has a guide section 43 extending in a relative displacement direction of the second body 60 with respect to the first body 40, in order to guide the second body 60 in the relative displacement direction.

Advantageously, the positioning part 84 forms the functional region of the coupling element 70 and the guide section 43, so that the shaping of the positioning part 84 permits a precise production with regard to low manufacturing tolerances and an advantageous guidance of the second body 60. Furthermore, the shaping has the result that no welding is necessary in the region of the coupling element.

In addition to the stabilization of body joints, embodiments shown in FIGS. 4a and 4b are also advantageous with regard to the support of sports equipment, in particular shoes, wherein the embodiments are advantageous especially in the case of shoelaces, lacing and adaptive soles.

FIG. 5 shows a further schematic representation of an apparatus 1.

The apparatus 1 comprises the receptacle 20, a first body 40 comprising a sealing section 80, a force transmission body 50, wherein the first body 40 and the force transmission body 50 may also be formed in one piece, a coupling element 70 which is not shown, and the second body 60.

In contrast to the previous embodiments, the sealing section 80 does not comprise a sealing lip 82, but only a positioning part 84. The sealing is carried out, for example, by means of the sealing section 80, in particular the positioning part 84. Alternatively, the first body 40 may also be formed in such a way that it provides a seal between a first chamber 25 and a second chamber 27, wherein a combined seal of sealing section 80 and first body 40 is also conceivable. If the first body 40 and the force transmission body 50 are formed in one piece, the sealing is accordingly possible via this. As a result of this seal, a fluid film would form between the inner surface of the receptacle 20 and the body formed for the sealing, wherein the fluid film would not influence the functionality of the apparatus 1. Without a sealing lip 82, a significantly advantageous continuous load stability can be achieved. Furthermore, the number of components required is reduced and the production is more cost-effective and simpler.

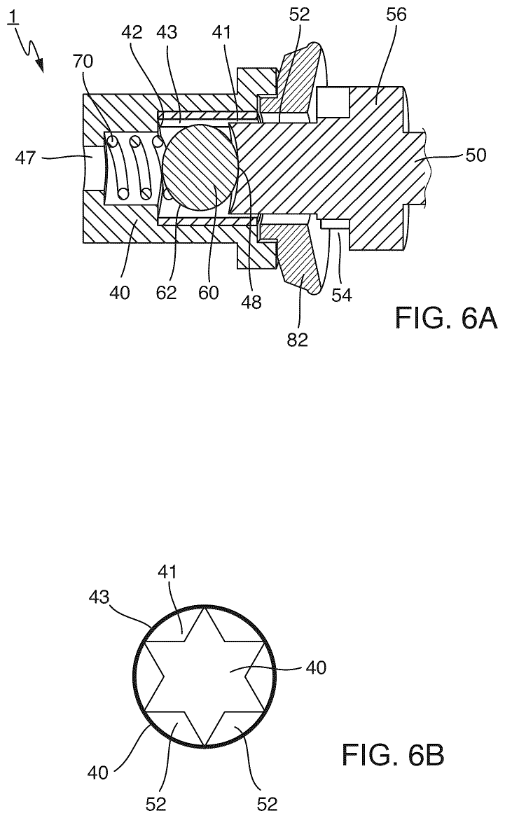

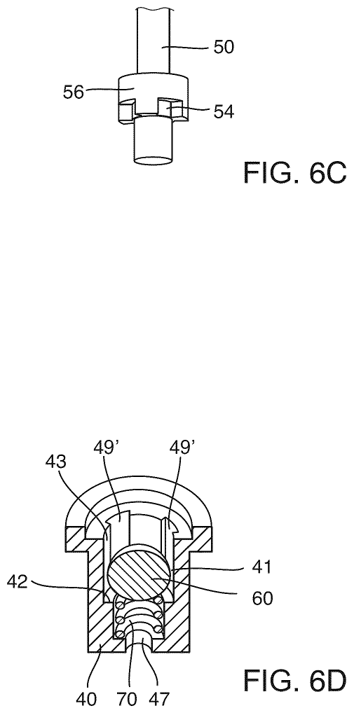

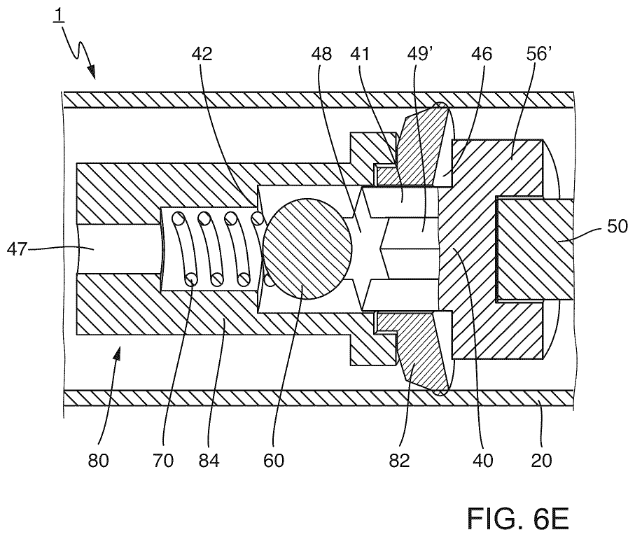

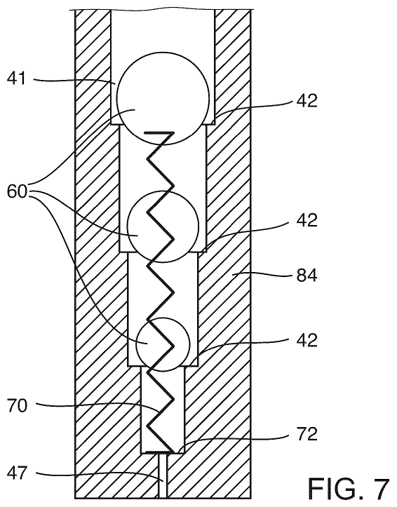

FIG. 6a shows a sectional representation through an alternative embodiment of an apparatus 1.

The apparatus 1 comprises, in addition to a receptacle 20 which is not shown, a second body 60, a force transmission body 50 and a first body 40, wherein the first body 40 comprises an adaptive sealing section 80.

The adaptive sealing section 80 comprises a positioning part 84 and a sealing lip 82, which is arranged fully circumferentially between the first body 40 and the inner surface of the receptacle 20 and makes contact with the latter, wherein the sealing lip is additionally pressed against the inner surface of the receptacle 20 by means of the filling medium 30 in the closed valve position. Furthermore, the first body 40 has a cross-sectional widening 56 in order to axially position the sealing lip 82 by means of the positioning part 84. The arrangement of the sealing lip is not restricted to the embodiment shown here.

Furthermore, the first body forms at least one passage opening 41 through which the filling medium 30 can flow, wherein the first body comprises a valve seat 42 in the region of the passage opening 41 and the second body 60 is displaceable received within the passage opening 41 of the positioning part 84. The passage opening 41 has a guide section 43 extending in a relative dis-placement direction of the second body 60 with respect to the first body 40, in order to guide the second body 60 in the relative displacement direction.

The force transmission body 50 has an end stop 48, wherein the force transmission body 50 has at least one recess 52, in the present exemplary embodiment a plurality of recesses 52, from the end stop 48 in the movement direction B up to the cross-sectional widening 56, so that the filling medium 30 can flow through the at least one recess 52 in the direction of the second body. The at least one recess 52 can be functionally considered as first end openings. Furthermore, the force transmission body has a plurality of grooves 54 in the region of the cross-sectional widening 56 in the direction of the first body.

In the present embodiment, the recesses 52 form a star shape, as viewed in cross section, as shown in FIG. 6b, wherein the disclosure is not restricted to the shape or number of recesses 52 described in this embodiment. Furthermore, it is possible to extend the recesses 52 in the axial direction into the cross-sectional widening 56, so that the recesses 52 replace the grooves 54 in the region of the cross-sectional widening 56.

Advantageously, the recesses 52 are distributed rotationally symmetrically and equidistantly on the circumference, so that a uniform incident flow of the second body 60 results.

In this case, a part of the force transmission body 50 facing the first body 40 is partially connected to the first body 40 via the guide section 43. The connection can be provided in a materially integral manner by means of welding, for example ultrasonic or laser welding, or as a force-fitting connection by means of pressing, wherein the force transmission body 50 does not make contact with the guide section 43 fully circumferentially.

This configuration permits a very compact shaping of the first body 40 and of the force transmission body 50, so that the distances between the inner surface of the receptacle 20 and the first body 40 or the force transmission body 50 permit a bending of the apparatus 1 about the connection in the region of the guide section 43 along the movement direction B.

FIG. 6c shows an alternative shaping of an apparatus 1 according to the disclosure.

In this case, the first body 40, is formed as a cylinder extending in the movement direction B in the region from the end stop 48 up to a cross-sectional widening 56. In addition, the cross-sectional widening 56 has a plurality of grooves 54 in the direction of the end stop 48.

As can be seen in FIG. 6d, at least one recess 49, in the present exemplary embodiment a plurality of recesses 49, is arranged in a guide section 43 and form the passage openings 41, wherein, analogously to the embodiment in FIGS. 6a and 6b, the force transmission body 50 is connected to the guide section 43 in a force-fitting or materially integral manner.

FIG. 6e shows a partial detail of a schematic sectional view of an alternative embodiment of a device 1 with recesses 49′ in the first body 40. The apparatus 1 comprises a first body 40 arranged in a displaceable manner in a receptacle 20 and a force transmission body 50 arranged on the first body 40 in the direction of the opening 22 of the receptacle 20. Furthermore, the first body 40 comprises an adaptive sealing section 80 comprising a positioning part 84 and a sealing lip 82. A second body 60 is displaceable received in the positioning part 84 and can be elastically coupled to the positioning part 84 via a coupling element 70.

The force transmission body 50 is connected to the first body 40, wherein the connection can be provided in a form-fitting manner, for example by means of welding, or in a force-fitting manner, for example by means of pressing.

The first body 40 has at least one recess 49′ from an end stop 48 in the movement direction B up to a cross-sectional widening 56′, so that the filling medium can flow through the at least one recess. In the embodiment shown, the at least one recess 49′ form at least one passage opening 41. For this purpose, the force transmission body 50 is connected to the adaptive sealing section 80, so that the second body 60 can be held in a starting position by means of the coupling element 70 against the end stop 48 of the first body 40. The connection between the first body 40 and the adaptive sealing section 80 can be carried out in a form-fitting manner, for example by means of welding, or in a force-fitting manner, for example by means of pressing.

FIG. 7 shows a sectional view of a positioning part 84 in an alternative embodiment. In the embodiment shown, the region of the positioning part 84 through which the filling medium 30 flows is constructed in a stepped manner. The stepped shape of the positioning part 84 narrows in the direction of the second end opening 47. This construction permits a plurality of second bodies 60 to be displaceable received in the positioning part 84. In this case, each second body 60, which simultaneously forms a valve body, has its own valve seat 42. In the present exemplary embodiment, three valves are formed. Analogously to the narrowing of the stepped shape of the positioning part 84, the surfaces 62 of the second bodies 60 decrease in the direction of the second end opening 47. In the embodiment shown, the second bodies 60 are spherical, wherein a different shaping is also possible. Furthermore, the second bodies 60 do not have to have the same shape. Further-more, the second bodies 60 can be elastically coupled to the positioning part 84 via a coupling element 70. As a result of the shaping, a passage opening 41 is produced which follows the stepped shape of the positioning part 84.

The three valves close in different flow states as a result of the surfaces 62 around which flow occurs of different sizes, so that a flow of the filling medium 30 through the passage opening 41 can be permitted or prevented in a stepped manner. As a result, for example, an adaptive sole can be provided which permits different bending stiffness to be present corresponding to the number of valves, with the result that the sole can be better adapted to the prevailing load state.

The disclosure is not restricted here to an embodiment with the number of valves shown in FIG. 7. Furthermore, in an alternative embodiment which is not shown, a plurality of second bodies 60 can be displaceable received in a first body 40.

To the extent applicable, all individual features which are illustrated in the exemplary embodiments can be combined with one another and/or replaced without departing from the scope of the disclosure.

### REFERENCE SIANS LIST

1 Apparatus
20 Receptacle
22 Opening
24 Interior
25 First chamber
27 Second chamber
30 Filling medium
40 First body
41 Passage opening
42 Valve seat
43 Guide section
44 Guide surface
45 Guide
46 First end opening
47 Second end opening
48 End stop
49 Recess
49 Recess
50 Force transmission body
52 Recess
54 Groove
56 Cross-sectional widening
56′ Cross-sectional widening
60 Second body
62 Surface
64 First surface section
66 Second surface section
70 Coupling element
72 Spring seat
80 Sealing section
82 Sealing lip
84 Positioning part
B Movement Direction

1. Apparatus for stabilizing body joints and/or for supporting sports equipment, comprising:
a receptacle, wherein the receptacle is filled with a filling medium,
a first body for interacting with the filling medium, wherein the first body is arranged displaceably in the receptacle,
a force transmission body for transmitting an external force to the first body (40),
a second body for interacting with the filling medium, which is arranged displaceably in the receptacle,
wherein the second body is coupled elastically to the first body via a coupling element,
wherein at least the first body has at least one passage opening through which the filling medium can flow, and
wherein the second body is received displaceably within the passage opening of the first body (40), wherein the first body comprises a valve seat in the region of the passage opening and the second body forms a valve body, so that a flow of the filling medium through the passage opening can be permitted or prevented depending on the valve position.

2. Apparatus of claim 1, wherein the passage opening has a guide section extending in a relative displacement direction of the second body with respect to the first body, in order to guide the second body in the relative displacement direction.

3. Apparatus of claim 1, wherein the inner surface of the passage opening has a guide in order to hold the second body centrally in the radial direction with respect to the inner surface of the passage opening.

4. Apparatus of claim 1, wherein the valve seat limits a relative displacement path of the second body with respect to the first body in the relative displacement direction.

5. Apparatus of claim 1, wherein the relative dimensions of the second body with respect to the passage opening are designed such that in the open valve position a fluid flow of the filling medium can be permitted in the region between the second body and the passage opening.

6. Apparatus of claim 1, wherein the second body is held in an open valve position by the coupling element in a starting position of the apparatus.

7. Apparatus of claim 1, wherein the coupling element is configured such that when physiological forces or speeds act on the apparatus the second body can be held in the open valve position by the coupling element against a flow force of the filling medium, and when non-physiological forces or speeds act on the apparatus the flow force of the filling medium can displace the second body into the closed valve position against a clamping force of the coupling element and hold it therein.

8. Apparatus of claim 1, wherein the second body is received in the passage opening of the first body such that filling medium can flow around at least one region of the surface of the second body in an open valve position.

9. Apparatus of claim 8, wherein the surface of the second body around which flow can pass extends from a first surface section, which faces at least one first end opening of the passage opening, to a second surface section, which faces at least one second end opening of the passage opening.

10. Apparatus of claim 1, wherein the second body the second body at least partially has a convex incident flow profile or a conical incident flow profile.

11. Apparatus of claim 1, wherein the second body is spherical.

12. Apparatus of claim 1, wherein the first body comprises a sealing section which is arranged between the first body and the inner surface of the receptacle in order to seal the first body with respect to the receptacle in the closed valve position.

13. Apparatus of claim 12, wherein the sealing section comprises a positioning part, wherein the passage opening is formed at least partially by the positioning part, and wherein the second body can be elastically coupled to the positioning part via a coupling element.

14. Apparatus of claim 1, wherein the force transmission body or the first body has an end stop, wherein the force transmission body has at least one recess from the end stop in the movement direction (B) up to a cross-sectional widening or the first body has at least one recess from the end stop in the movement direction (B) up to a cross-sectional widening and/or the first body has at least one recess extending from the valve seat along the movement direction (B) in the direction of the force transmission body, so that the filling medium can flow through the at least one recess.

15. Apparatus of claim 14, wherein the sealing section comprises a sealing lip, wherein the sealing lip can be pressed against the inner surface of the receptacle by means of the filling medium in the closed valve position and/or the first body divides a cavity of the receptacle into a first chamber and a second chamber, wherein the passage opening can provide a flow of the fluid medium between the first chamber and the second chamber in an open valve position.

16. Apparatus of claim 12, wherein the sealing section is an adaptive sealing section.

17. Apparatus of claim 2, wherein the inner surface of the passage opening has a guide in order to hold the second body centrally in the radial direction with respect to the inner surface of the passage opening.

18. Apparatus of claim 2, wherein the valve seat limits a relative displacement path of the second body with respect to the first body in the relative displacement direction.

19. Apparatus of claim 9, wherein the second body at least partially has a convex incident flow profile or a conical incident flow profile.

20. Apparatus of claim 9, wherein the second body is spherical.
